(19) United States
(12) Patent Application Publication
(10) Pub. No.: US 2009/0281532 A1
(43) Pub. Date: Nov. 12, 2009

(54) ARRANGEMENT FOR USE WITH A BALLOON ABLATION CATHETER

(75) Inventor: Vivek Reddy, Boston, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/116,703

(22) Filed: May 7, 2008

(51) Int. Cl. A61B 18/20; A61M 25/10; A61B 18/18; A61B 18/02

(52) U.S. Cl. 606/14; 604/101.02; 606/33; 606/21

(57) ABSTRACT

Along with the advancement of cryoablation procedures, there have been developments resulting in technology that can use catheters adapted with cryogenic balloons containing two non-compliant membranes. These cryogenic balloons do not contact significant portions of the targeted tissue, which can result in a longer than desirable ablation procedure. It may be advantageous for these cryogenic balloons to conform to the targeted tissue more effectively than prior advancements have enabled. According to an exemplary embodiment of the present invention, an expansion chamber can contain an inner membrane and an outer membrane. In an exemplary expansion membrane, the outer membrane can conform to an anatomical structure during an ablation procedure and the inner membrane can facilitate a selective expansion of the outer membrane. In a further exemplary embodiment of the present invention, the inner membrane can be provided with a plurality of membranes. In another exemplary embodiment of the present invention, an energy facilitating arrangement can be provided within the inner membrane. For example, cryogenic fluid, a laser or a RF coil can be provided as the energy facilitating arrangements within the inner membrane.

### FIELD OF THE INVENTION

The present invention relates to an arrangement for use with a balloon catheter system. More specifically, the present invention relates to an arrangement for conforming the balloon catheter to a targeted tissue to effectively ablate or otherwise affect the targeted tissue.

### BACKGROUND OF THE INVENTION

As in other fields of medicine, the use of cryogens (e.g., fluids with low operating temperatures) is being used in conjunction with catheter devices to ablate selected tissue areas within the body. Catheter-based devices may be used in various medical and surgical applications to provide a relatively non-invasive way of providing precise treatment to localized tissues that are otherwise inaccessible. Catheters may be easily inserted and navigated through the blood vessels and arteries, allowing non-invasive access to areas of the body with relatively little trauma. When used in conjunction with cryogens, catheters may be used to allow cryogens to flow within the catheter to selectively freeze, or “cold-treat”, and ablate targeted tissues within the body.

Catheter ablation devices are well-known in the art. In targeted tissue ablation, a cryogenic-catheter device can use energy transfer derived from thermodynamic changes occurring in the flow of a cryogen through the device to create a net transfer of heat flow from the targeted tissue to the device. Typically, this may be achieved by cooling a portion of the device to a very low temperature through conductive and convective heat transfer between the cryogen and the targeted tissue. The quality and magnitude of heat transfer may be regulated by the device configuration and control of the cryogen flow regime within the device.

By injecting the high pressure cryogen through an orifice of a catheter, a cooling effect can be achieved in a targeted area. Once injected from the orifice, the cryogen may undergo two primary thermodynamic changes: (i) expansion to low pressure and temperature through positive Joule-Thomson throttling, and (ii) a phase change from liquid to vapor, thereby absorbing heat of vaporization. The resultant flow of the low temperature cryogen through the device may act to absorb the heat from the targeted tissue, thereby cooling the targeted tissue to a desired temperature.

Once the cryogenic fluid is injected through an orifice, it may expand inside a closed expansion chamber, or balloon, which can be positioned proximal the target tissue. Devices with an expandable membrane, such as a balloon, may be employed as expansion chambers. In such devices, a refrigerant can be supplied through a catheter tube into an expandable balloon coupled to the catheter, where the refrigerant may act to both: (i) expand the balloon near the target tissue for the purpose of positioning the balloon, and (ii) cool the target tissue proximal to the balloon to cold-treat adjacent tissue.

One of the principal drawbacks to such a technique is that during the inflation phase the coolant may seep out of the balloon and enter into the bloodstream to cause significant harm. Therefore, if the balloon develops a crack, leak, rupture, or other critical structural integrity failure, the coolant may quickly flow out of the catheter. Another situation that may occur during the balloon deflation phase is that the balloon may adhere to the ablated tissue, also possibly causing severe damage.

To address these concerns a different expansion chamber may be provided to contain two expandable membranes or balloon, e.g., an inner membrane and an outer membrane. Both expandable membranes can be attached to the catheter with the inner membrane attached to the catheter such that it fills with the cryogenic fluid. A vacuum can then be applied between the inner membrane and the outer membrane such that if there is a leak or crack in the inner membrane, the vacuum will remove the cryogenic fluid, thereby reducing the risk of damage to the tissue and increase the effectiveness of the ablation process. Both membranes may be made of non-compliant materials with the limited flexibility such that there is a limited volume to which the membranes can expand.

Although this advancement may have the benefit of limiting the expansion chamber volume so that it does not expand beyond the size of a targeted tissue area, the rigid non-compliant nature of the membranes generally limit the expansion chamber's contact to the targeted tissue, thereby likely limiting the effectiveness of the ablation process. This may lead to ablation procedures that are longer than necessary and expose a patient to an increased risk of exposure to cryogenic fluid. This may occur after cryoablation or cryomapping. Cryomapping is a procedure that chills conducting target tissue to create a transient electrical effect. By temporarily chilling the target tissue, this procedure allows for a precise site confirmation in order to prevent an inadvertent ablation.

Accordingly, there is a need to overcome at least some of the deficiencies described herein above.

### SUMMARY OF THE INVENTION

To address and/or overcome at least some of the above-described problems and/or deficiencies as well as other deficiencies, exemplary embodiments of a system or arrangement can be provided. For example, such arrangements may include a first reservoir and at least one second reservoir. The first reservoir can conform to at least one section of an anatomical portion of a body. The second reservoir can limit the volume of the first reservoir. In a further exemplary embodiment of the present invention, the anatomical portion can be a pulmonary structure.

According to another exemplary embodiment of the present invention, the first reservoir may include at least one malleable portion which is capable of conforming to the at least one section. In a further exemplary embodiment, the second reservoir is provided within the first reservoir.

According to additional exemplary embodiment of the present invention, the second reservoir may contain a plurality of reservoirs. In a further exemplary embodiment of the present invention, at least one of the reservoirs may be proximal to at least another of the reservoirs. In a further exemplary embodiment, the first reservoir may be more malleable than the second reservoir.

According to another exemplary embodiment of the present invention, an outer surface of the first reservoir may conform to the at least one section and an inner surface of at least one portion of the first reservoir contacts the at least one second reservoir.

According to another exemplary embodiment of the present invention, an energy facilitating arrangement can be provided within the at least one second reservoir. In further exemplary embodiments of the present invention, the energy facilitating arrangement can be a cryogenic fluid, a laser or a RF coil.

### BRIEF DESCRIPTION OF THE DRAWINGS

The detailed description below will refer to the following illustrations, wherein like numerals refer to like elements, and wherein:

FIG. 1 is an exemplary illustration of an exemplary embodiment of the expansion chamber with an inner membrane and an outer membrane; and

FIG. 2 is an exemplary illustration of an exemplary embodiment of the expansion chamber with a plurality of inner membranes and an outer membrane.

FIG. 3 is an exemplary illustration of an exemplary embodiment of the expansion chamber with a laser energy facilitating arrangement provided in an inner membrane.

FIG. 4 is an exemplary illustration of an exemplary embodiment of the expansion chamber with a RF coil energy facilitating arrangement provided in an inner membrane.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF EXEMPLARY EMBODIMENTS

According to one exemplary embodiment of the present invention, an arrangement can be provided for improving a compliance of cryogenic expansion chambers. These cryogenic expansion chambers may be attached to catheters to be used in cryoablation of a targeted tissue.

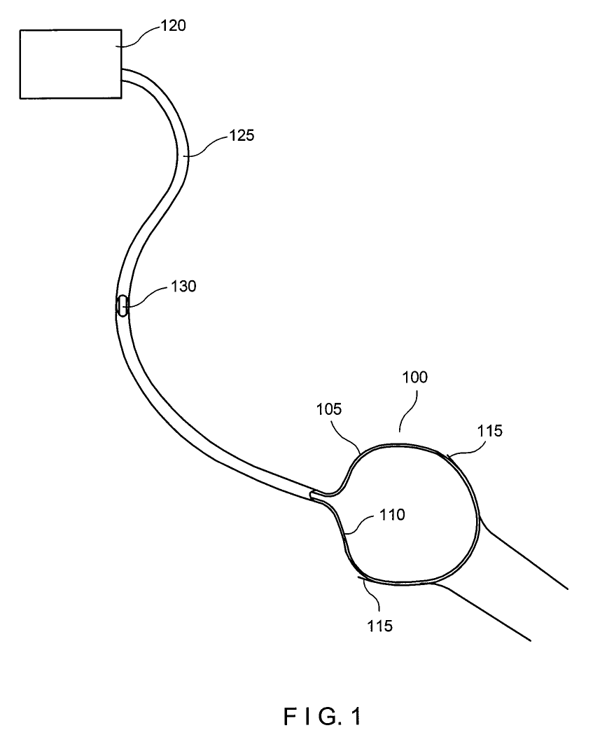

An exemplary embodiment of the arrangement according to the present invention can include an expansion chamber 100 containing two membranes. Referring to FIG. 1, an exemplary embodiment of the expansion chamber 100 may contain at least one outer membrane 105 and at least one inner membrane 110. The outer membrane 105 may be composed of a compliant material that can allow the expansion chamber 100 to contact the target tissue and secure or about the expansion chamber 100 to the target tissue 115 during ablation. For example, the outer membrane 105 may be composed of a compliant material that can be a rubber, latex, Teflon®, or any similar flexible material or polymer. The inner membrane 110 may be composed of a non-compliant material that can at least partially limit the maximum size of the expansion chamber 100. For example, the inner membrane 110 may be composed of a non-compliant material that is known in the art.

According to another exemplary arrangement of the present invention, the expansion chamber 100 can be attached to a catheter 120. For example, the catheter 120 can have a first connector 125 and a second connector 130. The first connector 125 can connect the outer membrane 105 to the catheter 120. In yet another exemplary embodiment of the present invention, the first connector 125 may be a vacuum line (e.g., 10 French lumen). The second connector 130 can connect the inner membrane 110 to the catheter 120. In still another exemplary embodiment of the present invention, the second connector 130 may be a coaxial line which is configured to allow for both a vacuum line (e.g., 8 French lumen), and an injection line (which may be used for the injection of cryogenic fluid).

For example, a cryogenic fluid may be injected into the inner membrane 110 from the catheter 120 through the second connector 130. Before the cryogenic fluid is introduced, however, a small amount of N₂O gas can be injected to slightly inflate the inner membrane 110. The first connector 125 and the vacuum line of the second connector 130 may draw any cryogenic fluid out of the inner membrane 110 and the outer membrane 105.

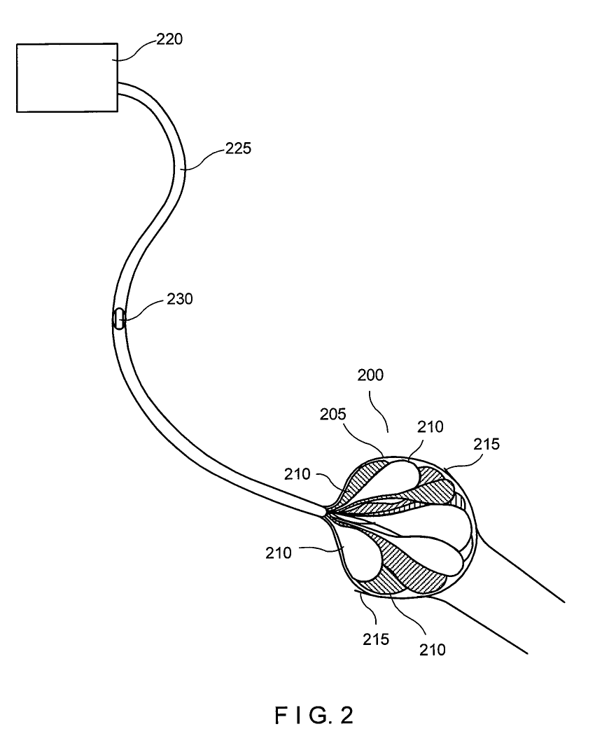

A further exemplary embodiment of the present invention can include an expansion chamber containing multiple membranes. Referring to FIG. 2, an exemplary embodiment of the expansion chamber 200 may contain at least one outer membrane 205 and a plurality of inner membranes 210. The outer membrane 205 may be composed of a compliant material that can allow the expansion chamber 200 to contact the target tissue and secure or about the expansion chamber 200 to the target tissue 215 during ablation. For example, the outer membrane 205 may be composed of a compliant material that can be a rubber, latex, Teflon®, or any similar flexible material or polymer. The plurality of inner membranes 210 may be composed of a non-compliant material that can limit the maximum size of the expansion chamber 200. For example, the plurality of inner membranes 210 may be composed of a non-compliant material that is known in the art.

In a further exemplary embodiment of the present invention, the expansion chamber 200 can be attached to a catheter 220. For example, the catheter 220 can have a first connector 225 and a second connector 230. The first connector 225 can connect the outer membrane 205 to the catheter 220. In an exemplary arrangement of the present invention, the first connector 225 may be a vacuum line, (e.g., 10 French lumen). The second connector 230 can connect the plurality of inner membranes 210 to the catheter 220. In yet another exemplary embodiment of the present invention, the second connector 230 may be a coaxial line allowing for both a vacuum line (e.g., 8 French lumen) and an injection line (e.g., which may be used for the injection of cryogenic fluid. In a further exemplary embodiment, the second connector 230 can separately provide medium to the plurality of inner membranes, which branch from the second connector 230.

The inner membranes 210 may be provided at different locations with respect to the extension of the second connector 230. This exemplary configuration of the inner membranes 210 can facilitate a selective expansion of the outer membrane 205 such that areas in the membrane, having different-sized cross sections, can be snuggly and securely abutted by the surfaces of the outer membrane 205.

For example, cryogenic fluid may be injected into the plurality of inner membranes 210 from the catheter 220 through the second connector 230. The first connector 225 and the vacuum line of the second connector 230 may draw any cryogenic fluid out of the inner membrane 210 and the outer membrane 205.

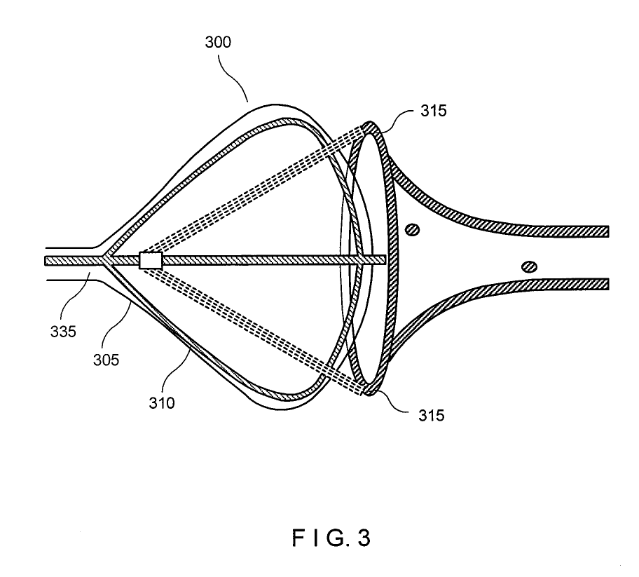

In further exemplary embodiments, other energy facilitating arrangements can be used instead of cryogenic fluid for tissue ablation. For example, additional energy facilitating arrangements such as a laser and a radio frequency (RF) coil could also be used for ablation. Referring to FIG. 1, in an exemplary embodiment of the present invention where the energy source is a laser, an optical fiber may be provided through the second connector 130. Further, a solution of D₂O can be provided through the second connector 130 to fill the inner membrane 110 with D₂O. The D₂O can provide an environment for the energy of the laser to be conducted through the expansion chamber 100 to ablate the target tissue 115. Referring to FIG. 3, an exemplary embodiment of the expansion chamber 300 is shown with a laser 335 as the energy facilitating arrangement. The inner chamber 310 can be filled with D₂O, and a vacuum can be applied to the space between the inner chamber 310 and the outer chamber 305, with the outer chamber 305 contacting (e.g. directly) the target tissue 315.

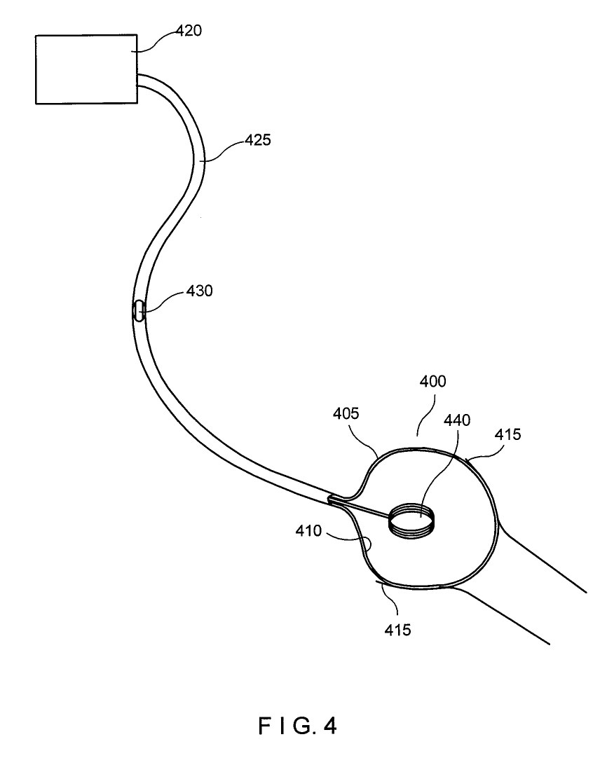

In a further embodiment of the present invention, a RF coil can be used as an energy facilitating arrangement in a saline solution environment. Referring to FIG. 4, an exemplary embodiment of the expansion chamber 400 is shown with a RF coil 440 as the facilitating arrangement. The inner chamber 410 can be filled with a saline solution, and a vacuum can be applied to the space between the inner chamber 410 and the outer chamber 405, with the outer chamber 305 contacting (e.g. touching) the target tissue 315.

Although the present invention has been described with respect to particular embodiments thereof, variations are possible. The present invention may be embodied in specific forms without departing from the essential spirit or attributes thereof. For example, although the present invention is illustrated with embodiments having an outer membrane composed of rubber, Teflon®, or latex, any malleable or flexible material or polymer may be used. Additionally, although the present invention is illustrated with an inner membrane or a plurality of inner membranes, any number of inner membranes may be used. It may be advantageous that the embodiments described herein be considered in all respect illustrative and not restrictive and that reference be made to the appended claims and their equivalents for determining the scope of the invention.

What is claimed is:

1. An arrangement, comprising:
a first reservoir being configured to conform to at least one section of an anatomical structure; and
at least one second reservoir being configured to limit a volume of the first reservoir.

2. The arrangement of claim 1, wherein the first reservoir comprises at least one malleable portion capable of conforming to the at least one section.

3. The arrangement of claim 1, wherein the at least one second reservoir is provided within the first reservoir.

4. The arrangement of claim 1, wherein the at least one second reservoir comprises a plurality of reservoirs.

5. The arrangement of claim 1, wherein the first reservoir is more malleable than the at least one second reservoir.

6. The arrangement of claim 1,
wherein an outer surface of the first reservoir conforms to the at least one section of anatomical structure, and
wherein an inner surface of at least one portion of the first reservoir contacts the at least one second reservoir.

7. The arrangement of claim 4, wherein at least one of the reservoirs is proximal to at least another of the reservoirs.

8. The arrangement of claim 1, wherein the anatomical structure is a pulmonary structure.

9. The arrangement of claim 1, further comprising:
an energy facilitating arrangement situated within the at least one second reservoir.

10. The arrangement of claim 9, wherein the energy facilitating arrangement is a laser.

11. The arrangement of claim 9, wherein the energy facilitating arrangement is a RF coil.

12. The arrangement of claim 9, wherein the energy facilitating arrangement is a cryogenic fluid.
